(19) United States
(12) Patent Application Publication
(10) Pub. No.: US 2009/0092693 A1
(43) Pub. Date: Apr. 9, 2009

(54) Platelet manipulation to prevent and treat endovascular disease and its sequelae, to prevent and treat arrhythmias and to prevent malignancy

(76) Inventor: Mady Attila, Kihei, HI (US)

Correspondence Address: Attila Mady, 105 Manino Circle, Suite 203, Kihei, HI, 96753, US

(21) Appl. No.: 11/868,393

(22) Filed: Oct. 5, 2007

(51) Int. Cl. A61K 36/8962; A61K 45/00; A61P 7/00; C12Q 1/02

(52) U.S. Cl. 424/754; 435/29; 514/789

(57) ABSTRACT

Reduction of platelet counts to below 150 thousand per cubic millimeter by pharmacologic, mechanical, combined mechanical and pharmacological, or other means for the purpose of reducing the incidence and severity of vascular disease in at risk populations, for the stabilization and reversal of said disease in patients already known to suffer from such disease, as well as for the purpose of preventing and/or reducing the incidence and severity of sequelae related to such disease, whether preclinical, subclinical or overtly manifested, or whether it is presently understood to be related to said vascular disease or not.

### TECHNICAL FIELD

The present invention relates to the prevention and treatment of vascular disease of all etiologies (with exclusions as specified in the lexicon above), as well as prevention and treatment of all sequelae of such disease.

### Preamble

A lexicon is deemed necessary due to the existence of prior art that is somewhat verbose, at times very concrete and specific, but overall self-contradictory and of little clinical utility. A list of exclusions to the scope of this application stemming from this prior art (U.S. Pat. Nos. 6,376,242, 6,585,995 & 7,022,521) shall therefore be provided in the Definitions section, followed by clarification of said exclusions and a demonstration of their limited clinical utility in the Summary and Detailed Discussion of the Invention.

### Definitions

“Vascular disease and its sequelae” includes atherosclerosis of all types and etiologies, intimal injury of any nature and etiology, other vessel wall injury, whether acute or chronic or whether involving large areas or microscopic segments and of any etiology. Vascular disease for the purposes of this patent may exclude acute vasoocclusive conditions and concentric intimal hyperplasia with luminal narrowing, as specified by U.S. Pat. Nos. 6,376,242, 6,585,995 & 7,022,521, but only for the specific matrix of interventions as specified in those two patents. Specifically, exchange transfusion or extracorporeal platelet manipulation in this setting shall be included in the definition, since only pharmacologic platelet reduction is included in the claims of this prior art.

“Vascular disease and its sequelae” also includes thrombotic, embolic or other particulate based conditions. Specifically, vascular disease shall also be deemed to include the specific cardiac diagnoses of Atrial Fibrillation and any other cardiac conditions leading to arrhythmias (such as cardiac non-compaction and ARVD—Arrhythmogenic Right Ventricular Dysplasia—to name a few).

“Vascular disease and its sequelae” is to include adult, foetal and congenital conditions.

“Vascular disease and its sequelae” is applicable to all species.

“Vascular disease and its sequelae” shall comprise all conditions that affect the function of all components of blood and lymphatic vessels and that result in or contribute to any harmful consequences to the organism, whether such a linkage may be presently recognized or not. One particular for instance would be Alzheimer's disease, which is probably caused by microvascular emboli and thrombosis and would thus fall in the category of “sequelae”.

“Vascular disease and its sequelae” also includes all pathologic states to which such disease might contribute, whether such linkage may currently be recognized or not. While understood that this list is not comprehensive, such sequelae would include obstruction, embolization, thrombosis, reduced blood flow, inflammatory states, chronic pain and reduced function to any body organs. Sequelae of vascular disease shall further include plaque and neurofibrillary deposits on macroscopic or microscopic scale, microvascular states, potentiation of metabolic disorders, tissue acidosis, as well as general ill-described malaise and failure to recover appropriately after stress conditions. Sequelae of vascular disease shall include a (past, present or future) history of cerebrovascular accidents, macrovascular or microvascular CNS disease, whether it be hypertensive, diabetic, or at present unknown etiology. Potential sequelae of vascular disease are to specifically include all malignancy states, whether overtly perfusion dependent or not. Sequelae of vascular disease are also to be assumed to include microvascular cardiac and peripheral disease (“Syndrome X”) and Burger's disease (endarteritis obliterans), as well as Alzheimer's and other microvascular CNS pathology. Additionally, sequelae of vascular disease for the purpose of this patent is include diseases such as ITP and HUS/TTP and their heretofore unrecognized analogues. Finally, sequelae of vascular disease will also include SIRS (systemic inflammatory response syndrome), as well as frank sepsis.

“Safe reduction of platelet counts” is to be understood to mean the reduction of platelet counts to levels that still permit adequate coagulation to prevent unduly excessive risk of major bleeding, and accomplished by means other than pathologic processes, whether acute or otherwise, that would lead to pathologic impairment of coagulation, as well as secondary disturbances of vascular integrity and function dependent on appropriate platelet function. “Safe reduction of platelet counts” shall further be construed to mean the employment of means designed to minimize the amount of platelet activation permitted during the process of platelet reduction (to avoid the elaboration and secretion of harmful substances into the serum), as well as to minimize the proportion of activated or near-activated platelets that would result from such a reduction method. IT SHOULD BE NOTED THAT HEMODIALYSIS PATIENTS WITH SPONTANEOUSLY REDUCED PLATELET COUNTS DO NOT MEET THIS SAFE REDUCTION THRESHOLD, SINCE THEIR PLATELET COUNTS ARE REDUCED DUE TO INCREASED TURNOVER AND DESTRUCTION AND THE REMAINING PLATELETS ARE OF ABNORMAL MORPHOLOGY AND SUBJECT TO DYSFUNCTION DUE TO UREMIA.

Adjunct modes of platelet inhibition and platelet function modulation are to include, but not be restricted to, use of acetyl salicylic acid, clopidogrel, persantine, EDTA, selective filters, irradiation, immunologic and chimeric immunologic therapeutics, IIb/IIIa inhibitors, acute thrombolytic agents, heparin, low molecular heparin, bivalarudin, hirudin and any other agents or interventions which have already been developed, or may be developed during the life of this patent, to have acute or chronic effects on platelet function and coagulation.

CNS—Central Nervous System.

### BACKGROUND OF THE INVENTION

Cardiac and vascular disease (and, specifically, atherosclerosis) have emerged as the number one killer of modern man. It is a disease of multiple risk factors, but without a proven etiology.

Regarding the currently prevailing theory of cholesterol metabolism disarray as the most important causative factor in the genesis of cardiac and vascular disease, it is remarkable that no one has ever proposed a logical justification for the existence of increased cardiovascular risk linked to the presence of increased cholesterol. The presence of such an ontological explanation would serve to bolster the selection of cholesterol as a causative factor, rather than merely as a factor associated with increased risk of vascular disease.

It is obvious, on the other hand, why animals of many species should have inordinately high platelet counts, in excess of what might be physiologic for long mammalian lives. Even while human beings had developed the genetic capacity for long lives in prehistoric times, trauma and infection were by far the most important mechanisms of demise. Life expectancies did not pass the three decade mark in many societies until the mid 19^{th} century and continue to remain around this figure in several undeveloped nations today. Given these factors, it is obvious that the ability to achieve rapid and effective hemostasis would far outweigh the need for reduced intravascular events in nature. It is thus logical to assume that if the coagulation cascade can be demonstrated to be a participant in the development of vascular disease—which it is known to be—then it is likely that the survival traits of rapid hemostasis have long been in conflict with the requirements of long term patency. In other words, the “coagulation thermostat” has been selected for maximum rapidity and efficacy and this is in direct conflict with what is required for optimal blood vessel patency.

It is recognized from the experience of the primary applicant of this patent, as well as multiple other cardiovascular physicians that no patient with normally functioning platelets and chronic counts significantly below normal has ever presented with cardiovascular disease, acute or chronic. In addition, it is known that patients with Glanzmann's thrombasthenia, a genetic abnormality preventing platelet activation, are also immune to this disease. It is further known that patients undergoing cardiovascular bypass (“perfusion”) and extra-corporeal membrane oxygenation for the purpose of cardiothoracic surgery experience a phenomenon known as “pump head”, heretofore of unknown etiology, but logically due to activated and microaggregated platelet clusters. Finally, it is fact that the process of hemodialysis activates platelets in a manner similar to the cardiovascular bypass machines—to the extent that most patients need to be heparinized—and that patients on hemodialysis have tremendously accelerated progression of atherosclerosis.

It is known that platelets are the most unstable of all cells in the body (Ref. 1, 2). It is also known that the presence of Angiotensin II in the circulation leads to the upregulation of NF Kappa B (Nuclear Factor Kappa B) and subsequent VCAM (Vascular Cell Adhesion Molecule 1) and PAI-1 (Plasminogen Activation Inhibitor 1) production (Ref. 3), both of which enhance the progression of atherosclerosis and even gross vascular deformities such as aneurysms. Similarly, the presence and induction of activated Protein C (APC) complexes also accelerate the progression of atherosclerosis. Controversy does exist whether genetically manipulated mice with increased PAI-1 expression showed more rapid spontaneous stepwise progression of atherosclerosis with some sources adamant in denial (Ref. 4, 5) and others equally categorical in support of a factorial correlation (Ref. 6). (Note, refer to Ref. 6 also for an excellent background bibliography on current thinking regarding genesis and progression of atherosclerosis). Endothelin is felt to be another compound with a role in the genesis of atherosclerosis. Von Willebrand factor is proposed to have a role in vascular disease development and manipulation of its levels is described by U.S. Pat. No. 7,192,914. However, regardless of the individual opinions regarding causative mechanisms, there is not a single individual working on the pathogenesis of atherosclerosis today who believes that cholesterol, whether in toto or a subset, is the sole or even predominant causative factor.

It is established that endovascular (i.e.: blood component) factors are more important than inherent vascular structural or chemical factors in the genesis of both micro and macrovascular disease. Specifically, contents and composition of circulating blood is more significant in the genesis of vascular pathology of all causes, whether atherosclerotic, inflammatory or other, than any structural, functional or chemical factors inherent to the vessel itself. Treatment therefore focuses on modulating the influences of these blood components, whether known or unknown.

The single most effective intervention in the prevention and treatment of vascular disease at this time remains Acetyl salicylic acid (Aspirin). Particularly for vasoocclusive disease, remarkably low doses of aspirin with remarkably low incidences of complications (less than 1% incidence of major haemorrhage) result in major outcome (death or further MI) of approximately 60%. This is even more remarkable in the light of the fact that we now know based on individual platelet function assays that up to 50% of patients treated with aspirin are non-responders, meaning genetically resistant to its acetylating effects.

It is also known that the earliest and most severe forms of atherosclerosis in the body occur at sites of maximum turbulence, such as in the aorta at the impact of a jet from a stenotic aortic valve. This mechanism of injury can only be due to intimal susceptibility or some unrecognized platelet effect, as platelets are the component of blood activated in such turbulent jets.

It is puzzling why there would be such a paucity of clinical and basic science research proceeding on the hypothesis that platelets themselves are an essential pre-requisite and final common pathway for all vascular injury. As a for instance are supplied three references (Ref. 7, 8, 9) addressing the correlation of megakaryocyte dysfunction in diseases states (specifically, diabetes), the effect of fat ingestion on platelet function and the platelet size (as a marker of platelet activation) as a positive risk for acute myocardial events (i.e.: as a marker of acute endovascular injury), respectively. Of note, however, there has not been a single study to examine whether safe reduction of platelet counts to minimum levels permissible without undue increase of major bleeding might not reduce the incidence of various vascular pathology.

Given the above clinical and basic science, as well as the fact that aspirin's effects are almost exclusively confined to the platelet component of blood, it is believed self-evident that research should concentrate on the structure and function of platelets, as well as the manipulation of said structure and function as the key to solving the riddle of the overwhelming majority of vascular diseases. A logical simple early solution to what shall indubitably prove to be an exceedingly complex mechanism and array of malfunctions is to safely reduce the number of provoking entities, namely platelets, available to induce injury.

Further, there is evidence that the initial establishment of tumors is not possible without angiogenesis. In addition, there is evidence that thrombin receptors have a role in this initial angiogenesis. Platelets have a role in modulation of thrombin receptors. Reduction of platelets to safe levels would affect thrombin receptor levels.

Finally, for anyone in doubt of the veracity of the potency of platelet effect on fibroblast growth and selected cellular activity, refer to U.S. Pat. No. 5,165,938. Platelet extracts cause visible increase in fibroblast activity that can be demonstrated through gross cell culture assays. Platelets (and compounds released by platelets) have a similar inflammatory effect on white cells and multiple other blood and vascular components.

Priority in art is claimed given that no person—physician, biological scientist, or otherwise—has ever documented in a public or restricted but independently verifiable forum the understanding that vascular pathology in a very wide spectrum is either markedly reduced or completely absent in the presence of safely reduced platelet counts.

### SUMMARY OF THE INVENTION

Reduction of platelet counts to below 150 thousand per cubic millimeter by pharmacologic, mechanical, combined mechanical and pharmacological, or other means for the purpose of reducing the incidence and severity of vascular disease in at risk populations, for the stabilization and reversal of said disease in patients already known to suffer from such disease, as well as for the purpose of preventing and/or reducing the incidence and severity of sequelae related to such disease, whether preclinical, subclinical or overtly manifested, or whether it is presently understood to be related to said vascular disease or not.

### DETAILED DESCRIPTION OF THE INVENTION

Given the above background, it is thus concluded that platelets are a pre-requisite for vascular injury. Platelets are the common final pathway for all such injury and can therefore be termed to be the true causative factor for such injury. This is a unique novel improvement on the already universally accepted maxim that cholesterol and other presently hotly pursued factors are merely contributing or incidental factors.

Since bleeding rates in people with normally functioning platelets are not increased until platelet counts are significantly decreased below normative levels, patients can tolerate marked reductions in platelet counts FROM WHAT ARE CONSIDERED NORMAL LEVELS without any significant morbidity. Specifically, people with normal platelet function can tolerate platelet counts in the range of 50000 platelets per cubic millimeter without any increased risk of bleeding.

Additionally, the need to temporarily remove and then re-introduce platelets to avoid activation in certain chronic disease states (such as during hemodialysis) has also not been recognized to date. Nevertheless, advances in technology have made such interventions plausible. The present invention capitalizes on these advancements and attains heretofore implausible objectives by prescribing a radical departure from current methods in the prevention and treatment of endovascular cardiovascular and neurovascular disease.

Rather than focusing on loosely associated risk factors never proven to directly cause atherosclerosis, the only plausible etiology has been deduced from existing clinical, basic science research and pharmacologic treatment data. This causative factor, namely the presence of an unnecessarily high density of platelets subject to activation in the endovascular space, as well as in reserve in various extravascular tissues, can now be treated with a variety of interventions, as appropriate for specific disease conditions.

This reduction can be achieved through pharmacologic or mechanical means (“mechanical means” to subsume irradiation and extracorporeal interventions, such as plateletpheresis, centrifugal separation and exchange transfusion, as well as any current and future available interventions that are not primarily pharmacologic). This reduction can be achieved on an ongoing or episodic basis, such as coupled with episodes of hemodialysis. This level of reduction can be precisely regulated to optimal levels as determined based on the projected severity of the pathology to be avoided, thus requiring a flexible definition of “safe reduction of the number of platelets”.

Acceptable rates of major bleeding would thus also depend on the nature of the projected or already existent pathology to be addressed. Reduction in platelet counts can be combined with selective platelet inhibition to reduce the activity of the platelets that do remain, as well as to block the effect of substances secreted by the platelets. Platelet function and coagulation assays can be used as an adjunct to further refine the precision of platelet function manipulation.

Generally, platelet counts are envisioned to be reduced to between 50000 to 150000, based on the nature of the pathology to be avoided or treated. Advanced atherosclerotic disease, coronary or peripheral vascular, will require more aggressive reductions. Early and advanced CNS microvascular/microfibrillary disease would similarly mandate very aggressive management.

Haemodialysis patients should be treated through combined pharmacologic and mechanical separation means. It is envisioned that the long term future of haemodialysis shall be in the more physiologic, longer and less intensive forms, such as ultrafiltration and SLED (sustained low efficiency dialysis). While these might be less irritating to platelets, and therefore more benign to the endothelium, it is nevertheless prudent to minimize the number of platelets transiting through the haemodialysis circuitry. It is thus proposed that all forms of mechanical haemodialysis shall require an extracorporeal separation stage with or without chronic platelet inhibition and chronic platelet level reduction as part of the haemodialysis process.

Adjunct modes of platelet inhibition and platelet function modulation are outlined above in the lexicon section and may include, but are not restricted to, use of acetyl salicylic acid, clopidogrel, persantine, EDTA, selective filters, irradiation, immunologic and chimeric immunologic therapeutics, IIb/IIIa inhibitors, acute thrombolytic agents, heparin, low molecular heparin, bivalarudin, hirudin and any other agents known to have acute or chronic effects on platelet function and coagulation.

### DISTINCTION FROM PRIOR ART

As noted above, U.S. Pat. Nos. 6,376,242, 6,585,995 & 7,022,521 specifically refer to the use of anagrelide and other pharmacologic agents to include MPL pathway inhibitors in acute vaso-occlusive crisis.

U.S. Pat. Nos. 6,376,242, 6,585,995 & 7,022,521 comprise 10, 99 and 47 claims, respectively. These claims are very specific and categorical. The use of the verb “is” in each claim, as well as the extraordinary number of very specific claims, make it clear that the claims subsume the full spectrum of envisioned clinical scenarios.

Unfortunately, there appear to be several fatal flaws in the prior art. The first issue is the reference specifically to vaso-occlusive disease. The invention as described mandates the presence of a significant degree of endovascular obstruction. One claim does allude to a scenario “wherein the patient may appear to be healthy” (i.e.: presumably asymptomatic). This claim however is specifically referenced to and is subordinate to the primary claim presuming the pre-existence of clinically detectable vaso-occlusive disease. This combination therefore excludes the vast majority of vasculopaths due to the Glagov phenomenon. The Glagov phenomenon states that vessels initially remodel concentrically, meaning expanding outwards from the center and preserving luminal anatomy. For this reason the phrase “intimal hyperplasia” also becomes unduly restricted (and therefore rendered clinically useless) by the claims of the patent, as most initial remodeling occurs behind the intima and in many cases without significant disturbance of the intima.

In case there might be the slightest controversy regarding the intent of the prior art, the initial claim specifically refers to “patients who need of such a treatment” as referenced to the pathological condition of an acute vaso-occlusive crisis. These claims therefore mandate that one must first somehow demonstrate the existence of stenosis or thrombosis or some other mechanism of occlusion, even though the “patient may appear healthy”. The verbiage as written demonstrates disregard of the greatest difficulty in cardiovascular medicine, namely the complexity of accurate patient triage for specific treatments and interventions. This triage is an overall estimation of the probability and projected severity of future negative outcomes and integrates anatomic, physiologic, functional, genetic, behavioral, historic, as well as objective testing data.

Extraordinary resources are invested into the construction and execution of massive trials for the purpose of elucidating appropriate sub-stratification of candidates for applicable potential treatment modalities. This is because it has been found that blanket application of today's potent medicines and interventions is likely to be of neutral outcome at best, particularly in acute settings. It is known that particular care must be exercised when it comes to initiation of therapy in the setting of asymptomatic endovascular disease. Interference in asymptomatic disease states with any but the most indolent treatment modalities has consistently resulted in increased incidence of negative outcomes.

Through our understanding of the Glagov phenomenon, as well insights regarding formation of collateral circulation and other compensatory mechanisms in chronic endovascular obstruction, we now know that only a miniscule subset of patients are impacted primarily by slowly progressive obliteration of vessel lumen. Even hemodynamically significant endovascular stenosis (specifically defined as >>50% by invasive and interventional cardiologists) is usually well tolerated, as long as there is no acute plaque rupture. The above mentioned clinical trials have confirmed the suspicion that attempts to mechanically interfere with this heretofore inexorable process is only useful in the acute setting (i.e.: acute occlusion due to plaque rupture and thrombus).

For this reason, the concept of “vaso-occlusive disease” is outmoded and no longer utilized. The body has too many adaptive mechanisms to compensate for chronic (slowly progressive) stenosis for this concept of slow occlusion as a means of functional reduction to have any significance. Unless (as noted below) one proceeds on the assumption that this chronic progressive obstruction is reversible.

The term vaso-occlusive event (or crisis) is therefore reserved for the acute setting. As used in the prior art, the concept of vaso-occlusive event is understood constitute the rupture of a susceptible plaque with the formation of a locally obstructive thrombus. Such episodes, however, are overt and acute and are detected because of patient symptoms. Patients with vaso-occlusive episodes do not “appear healthy” (if they did, the vessel occlusion would not be diagnosed). The concept of “apparently healthy” patients with “vaso-occlusive” events is nonsensical, since there is no way of identifying such patients. It is not clinically practical to propose attempting to identify patients who appear healthy and yet are having “vaso-occlusive events”, in order to determine that they should be subjected to urgent and acute platelet reduction. Even if absurd resources were committed to such an undertaking, the Glagov phenomenon tells us that if all early vasculopaths were subjected to invasive angiograms (still the most sensitive technique for detecting vascular stenosis) or equivalent imaging techniques (echocardiogram, vascular ultrasound, CT angiogram or MR angiogram), none of these patients would fall in this early stage into the “vaso-occlusive” category (since their luminal diameter and morpohology would be preserved).

CT angiograms specifically can detect changes in the vessel wall, but this qualifies as vascular pathology, not asymptomatic “vaso-occlusive events”. There is no way and likely shall remain no way of identifying asymptomatic patients undergoing vaso-occlusive crises (if indeed such a thing is possible). Asymptomatic patients are triaged for therapy based on exercise tolerance and estimated risk calculated from such factors as family history and cholesterol levels. These criteria in turn have no correlation to the concept of “vaso-occlusive event”, except to assess its downstream risk. And this possible eventuality has no correlation to the scenario outlined in the prior art as cited here:

“1. A method for treating a subject to inhibit a vaso-occlusive event comprising administering to a subject in need of such treatment an agent that reduces platelet count in the subject in an amount effective to reduce platelet count in the subject to at least a low normal level . . .
13. The method of Claim 1, wherein the subject is otherwise free of symptoms calling for treatment with the agent.
14. The method of Claim 1, wherein the subject is apparently healthy.”

The prior art must therefore be interpreted to restrict its scope to acute and detectable, or somehow very strongly suspected, vaso-occlusive episodes. The purpose of reducing platelet counts is merely to diminish the severity and duration of the vaso-occlusive crisis. Whether symptomatic or not, however, physiology tells us that ischemic episodes are rapid and dramatic. This in turn means that powerful extracorporeal (non-pharmacologic) platelet reduction techniques are required to achieve a sufficiently rapid and intense drop in platelet levels to stand any chance of a clinically relevant impact. (a novel platelet separation is proposed in a separate, but concurrent, patent application).

Due to the delays of onset of action, a pharmacologic platelet reduction (or, more specifically, platelet reduction by pharmacologic inhibition of platelet production and/or release into the bloodstream) is unfortunately unlikely to be of any use in clinical practice in the setting of acute vaso-occlusive disease. But the claims of the prior art specifically exclude non-pharmacologic intervention as a means of platelet reduction in the setting of acute vaso-occlusive events. Specifically, the claims are very explicit in prescribing only pharmacologic intervention to inhibit megakaryocyte production of platelets. While there is some allusion to such methods in the body of the text, their explicit exclusion in the claims means that these embodiments are not protected under this prior art.

Additionally, the claims also omit to mention the downside of excessive platelet depletion inherent in the rapid platelet adjustments required in the acute care setting. It is for this reason that this patent emphasizes the need for ongoing accurate monitoring of treatment effects. In this vein, a further significant issue is the infinite array of ranges supplied for possible platelet reduction targets. The only specific reference is to reduction of platelet counts to “at least low normal”. This vagueness demonstrates clearly the lack of insight regarding the pathogenetic nature of even normal platelet counts. The present invention prescribes a much more precise and rational method for achieving the lowest possible tolerated platelet counts, which are requisite for minimizing the risk of downstream events.

But the most significant deficit of the prior art is the lack of insight regarding platelets as a causative factor (or, possibly, the primary causative factor) of atherosclerosis itself. The most significant advancement in this application is the promotion of platelets from a bystander status in vascular disease to an important (and possibly primary) causative factor of atherosclerosis, endovascular disease and a significant range of other vascular pathologies and related sequelae. Platelets are more than a passive component of acute thrombi and emboli, they are pathogenic in their own right. We have far more platelets than we need to safely go about our daily routine and we can now safely manipulate these platelet levels with a minimum of complications.

Since platelets are only one cofactor in acute vaso-occlusive disease, their manipulation will probably not solve the issue of ischemic crisis. Further, it has already mentioned that if manipulation of platelet concentrations is proposed, it will not be useful via MPL pathway inhibitors, anagrelide, or any other agents targeted to the reduction of platelet production. But there is overwhelming clinical evidence to suggest that chronic manipulation of platelet levels, as in the case of aspirin use, is highly effective. Therefore, as with aspirin, intervention should be initiated early (possibly as early as in childhood in patients with known genetic risk), applied widely and with the understanding that it would be most effective in the setting of prevention.

As a footnote, the proposed advancement in the state of the art may incidentally (ironically) resurrect the concept of vaso-occlusive disease. While this concept is known to be useless in the current clinical milieu, the ability to reverse advanced disease would once again make useful the detection of luminal irregularities as a marker of early endovascular disease. The removal of excessive platelets and chemicals released during their activation might make it possible to reverse disease that at present the body has to compensate for by indirect means.

In conclusion, the prior art as outlined in U.S. Pat. Nos. 6,376,242, 6,585,995 & 7,022,521 suffers from critical omissions, inconsistencies and limitations of scope that render its prescriptions useless in terms of clinical utility. While the three cited patents comprising the most closely applicable prior art do contain a large number of very specific claims, they restrict their claims to discrete vaso-occlusive episodes and to pharmacologic platelet manipulation through inhibition of platelet production and/or release. These claims are vague regarding optimal platelet count targets, are too specific in referring exclusively to pharmacologic means of platelet count manipulation in this acute care setting and encompass only a minute subset of the full spectrum of pathologies that are likely to benefit from platelet reduction therapy, acute or chronic. The improvements proposed herein are by no means obvious and are based on the fundamental insight that platelets are not merely a component of obstructive thrombi due to atherosclerosis, but are themselves either a contributive or outright causative factor even (at normal counts) of very wide range of cardiac, microvascular and macrovascular pathologies.

Finally, one additional patent, U.S. Pat. No. 7,192,914 proposes to prevent atherosclerosis through decreasing circulating von Willebrand factor levels (and, specifically, von Willebrand factor multimers). It is stated that von Willebrand factor is directly responsible for the production of atherosclerosis. However, there is no bench or clinical data supplied to substantiate this hypothesis.

While it is a refreshing departure from the current unsubstantiated dogma that something other than cholesterol may be the primary cause of atherosclerosis, U.S. Pat. No. 7,192,914 fails to recognize that reduction in von Willebrand factor translates directly into platelet reduction. This platelet reduction is far more likely to be responsible for the lowered incidence of atherosclerosis than the decrease in the von Willebrand factor itself. Lowering platelet levels directly is therefore likely to result in a much greater impact than addressing von Willebrand factor levels. Reduction of von Willebrand levels in turn may or may not result in additional inhibition of atherosclerosis and vascular disease.

### DESCRIPTION OF THE SPECIFIC EMBODIMENTS

Several methods are currently available to reduce platelet counts in human beings. The most obvious and widely used is plateletpheresis, a centrifugal elimination of platelets from the blood. This is relatively atraumatic, but does result in the activation of some platelets.

Filters and adsorbent substrates are also used to eliminate platelets from blood. These would be considered inadvisable given the amount of platelet activation prior to sequestration from the blood stream.

Long term ideal separation of platelets from blood would probably require complete blood decomposition and removal of platelets from serum as the last component. This sort of mechanical separation and reconstitution would serve as the mainstay for patients who already have advanced vascular disease and who also have thrombocytopenia (such as hemodialysis patients).

Anagrelide is a substance known to inhibit the production of platelets. Currently it is only used to reduce counts in patients with thrombocytosis (or high platelet counts). In extremely rare cases it has been reputed to precipitate acute coronary syndromes, including heart attacks. On the face this might therefore be considered a second line agent. However clinical experience of use of Anagrelide concerns only patients with ELEVATED platelet counts—who are already at excess cardiovascular risk.

The administration of Anagrelide is known to reduce platelet counts without any significant impact on the morphology and activity of the platelets produced by megakaryocytes influenced by it. This means that reduction below population norms (specifically 150000 platelets per cubic millimeter of blood) is unlikely to cause increased intravascular events, unless it is discovered that platelet size to below population norms (specifically 150000 platelets per cubic millimeter of blood) would result in increased platelet size and activation (increased platelet size in itself would imply that a larger number of activated platelets are produced by the marrow). However, such a finding would easily be remedied by the use of aspirin, clopidogrel or a similar antiplatelet agent.

Similar, but more effective and/or less harmful medicines such as monoclonal agents or more specific bone marrow modulators, however, might emerge in the future and provide viable means of pharmacologic reduction. This patent is to address their use as platelet reducing agents for the specific purpose of addressing vascular injury and its sequelae as outlined in other parts of this patent application.

Further possibilities of platelet reduction might include whole blood removal (phlebotomy), selective malnutrition, or monoclonally or passively activated platelet removal via adherence or macroclumping and filtration and then subsequent plasma treatment if necessary. Any process proven of low morbidity and high efficacy in this regard would be viable.

The addition of a platelet pheresis stage to a perfusion circuit, extra corporeal membrane oxygenator and hemodialysis circuit is an application that requires a mechanical application. This mechanical separator would have to be more temporally efficient then current plateletpheresis equipment, possibly necessitating the addition of a passive or active platelet activating sequence and subsequent cluster filtration and plasma treatment independent of the perfusion circuit, extra corporeal membrane oxygenator or hemodialysis circuit.

A novel platelet separator utilizing electric fields and/or mixed phase reactions is proposed concurrently with this patent application. It is hoped that this technology will enable atraumatic separation of platelets from blood with sufficient efficiency to both prevent platelet-related morbidity and to permit use in real time applications.

Although the foregoing invention has been described in some detail by way of illustration and example for purposes of clarity of understanding, it will be understood that certain changes and modifications may be practiced within the scope of the appended claims. Thus, in accordance with the foregoing disclosure, the invention is not to be limited by the examples and descriptions herein, but is to be determined in scope by the claims provided.

1. Initial assessment of platelet counts, as well as other platelet and coagulation parameters as desired (platelet activity, bleeding time and coagulation assays) and as permitted by available resources in all patients subject to cardiac and vascular pathology and its sequelae

2. The safe reduction of said platelet counts of claim 1 by mechanical, pharmacologic or other means to levels significantly below the individual's physiologic norms, to be targeted ideally significantly under what is currently considered to be the lower limit of population norm (150000 platelets per cubic millimeter), for the purpose of avoiding and/or treating cardiac and vascular pathology, as well as its sequelae, as outlined in the lexicon below

3. The possible further gradual safe reduction, if indicated or deemed advisable, of said platelet counts of claim 1 to minimum levels tolerated without any episodes of major bleeding, depending on the nature of the cardiac and/or vascular pathologies contemplated or present

4. The consideration of using bleeding time and the most accurate and analytical available platelet count and function assays, as well as coagulation indices, available to the clinical practitioner and patient, to assess the efficacy and risks of said treatments based on manipulating the platelet counts of claim 1.

5. The consideration of periodic objective assessment of the efficacy and possible complications of said treatment resulting from the reduction of the platelet counts of claim 1, particularly in patients with known existing pathology.

6. The consideration and possible inclusion of additional modes of platelet inhibition and platelet function modulation to enhance and/or refine the precision of overall platelet activity control resulting from reduction of the platelet counts of claim 1.

7. The reduction of the platelet counts of claim 1 before, during or after renal replacement therapy to contain the damage done by activated platelets and thus avoid and/or treat cardiac and vascular pathology, as well as its sequelae

8. Deliberate pre-operative, peri-operative and post-operative regulation of platelet counts and/or platelet activity of claim 1

9. Platelet reduction in the setting of acute vasoocclusive events (cardiovascular, vascular and CNS) resulting either from obstructive luminal stenosis and/or intimal hyperplasia resulting in thrombotic or embolic luminal endovascular obstruction by non-pharmacologic means and according to of the process described in claims 1-7

10. The concurrent administration of onion extracts via the gastrointestinal tract, endotracheally, parenterally or transcutaneously to enhance the effects of the process described in claims 1-7

11. The use of chronic platelet reduction therapy described in claims 1-7 for the prevention of cancer in at risk individuals.

12. Deliberate reduction of platelets, whether by mechanical, pharmacologic or any other means, in conjunction with (i.e.: prior to, during or immediately after) cardioscular bypass surgery, blood scavenging during any major surgery, or renal replacement therapy.
